(19) United States
(12) Patent Application Publication
(10) Pub. No.: US 2025/0099109 A1
(43) Pub. Date: Mar. 27, 2025

(54) PRESSURE APPLICATION SYSTEM

(71) Applicant: ARMR Systems, Inc., Baltimore, MD (US)

(72) Inventors: Chibueze Joseph Ihenacho, Wheaton, MD (US); Thomas Trevor Bludis, Baltimore, MD (US); Andrew James Stephens, Frederick, MD (US)

(21) Appl. No.: 18/821,532

(22) Filed: Aug. 30, 2024

Related U.S. Application Data
(60) Provisional application No. 63/537,806, filed on Sep. 11, 2023.

(51) Int. Cl. A61B 17/132; A61B 17/00

(52) U.S. Cl. CPC A61B 17/1325; A61B 2017/00407

(57) ABSTRACT

Methods and systems for pressure application are provided. A pressure application system comprises a base assembly having an upper profile and a lower profile, a chassis coupled to the base assembly, a ratchet, a handle coupled with the ratchet, and at least on strap retention mechanism coupled to the chassis. The ratchet extends through the chassis and least partially in the base assembly. The handle is coupled with the ratchet such that a movement of the handle moves the ratchet. The at least one strap retention mechanism is configured to receive a strap retention mechanism.

### CROSS-REFERENCE TO RELATED APPLICATIONS

This application claims priority U.S. Provisional App. No. 63/537,806, filed on Sep. 11, 2023, which is herein incorporated by reference in its entirety.

### TECHNICAL FIELD

The present disclosure is directed to a system or device configured to apply pressure at a desired location on a body of a user. The pressure can be applied to reduce or stop bleeding, tighten a grip, or hold, on the area, or stabilize the area.

### BACKGROUND

Pressure can be applied to various areas of a human subject to facilitate tightening, local stabilization, or reduce or prevent bleeding. A common device used to achieve such pressure is tourniquet. A tourniquet can be used to stop or reduce bleeding that can occur as a result of an injury. The tourniquet can be used to control venous and arterial circulation to the site of the injury. The tourniquet can control the blood flow by applying pressure to the tissue underlying the tourniquet. The applied pressure can occlude the vessels and prevent or reduce flow therethrough. Conventional tourniquets, among other pressure application devices, can be unpredictable and difficult to apply the target pressure for the desired results.

### SUMMARY

In some aspects, a pressure application system may include a base assembly with an upper profile and a lower profile, a chassis coupled to the base assembly, and a ratchet extending through the chassis and at least partially into the base assembly. The system may also include a fin extending from a surface of the ratchet, a handle coupled with the ratchet and engaging a primary screw of the ratchet, and at least one strap retention mechanism coupled to the chassis configured to receive and retain a strap. Rotation of the handle below a torque threshold may cause the fin to rotate and wind the strap around the ratchet to tension the strap, while rotation above the torque threshold may translate the ratchet axially to extend the lower profile away from the upper profile.

In some aspects, the chassis may be movably coupled with the base assembly to allow movement of the base assembly relative to the chassis. This movable coupling may facilitate adjustment and positioning of the pressure application system.

In some implementations, the ratchet may be movably coupled with the chassis to allow movement of the chassis relative to the ratchet. This configuration may provide flexibility in the operation of the pressure application system.

The chassis may include a chassis aperture configured to receive at least a portion of the ratchet. This aperture may allow the ratchet to extend through the chassis while maintaining proper alignment and functionality.

In some aspects, the chassis may include at least one engagement feature configured to engage the at least one strap retention mechanism to retain the strap retention mechanism in a latched position. This engagement feature may help secure the strap in place during use of the pressure application system.

The at least one strap retention mechanism may include a latch configured to be received by the at least one engagement feature. This latch mechanism may provide a secure connection between the strap retention mechanism and the engagement feature.

In some implementations, the ratchet may include a body defining a body aperture extending axially through at least a portion of the body. This body aperture may accommodate other components of the ratchet mechanism.

The pressure application system may include a spool disposed at least partially in the body aperture of the ratchet. This spool may facilitate the winding and tensioning of the strap.

In some aspects, the spool may include a spool aperture extending axially therethrough, wherein the primary screw is partially disposed in the spool aperture. This configuration may allow for the translation of rotational motion into linear motion for extending the lower profile.

In some aspects, a method of using a pressure application system may include orienting the system at a location on a user's body, applying a strap around the location, and rotating a handle to tension the strap to reach a torque threshold. The system used in the method may comprise a base assembly, chassis, ratchet, fin, handle, and strap retention mechanism similar to those described above. The method may involve extending the strap over the fin from one side of the chassis to the other and engaging portions of the strap with strap retention mechanisms on each side.

In some aspects, the method may include rotating the handle beyond the torque threshold to extend the ratchet. This additional rotation may cause the ratchet to translate axially, extending the lower profile away from the upper profile and applying increased pressure to the target area on the user's body.

The method may also include a step of reversibly rotating the handle and flipping a lever to release the tensioning of the strap. This feature may allow for quick and easy removal or adjustment of the pressure application system when needed.

In some implementations, applying the strap around the location of the body may involve extending the strap from a first side of the chassis, over the fin, to a second side of the chassis. This configuration may ensure proper alignment and tension of the strap for effective pressure application.

The method may further include engaging a first portion of the strap with a first strap retention mechanism disposed on the first side of the chassis, and engaging a second portion of the strap with a second strap retention mechanism disposed on the second side of the chassis. This dual engagement may help secure the strap in place and maintain consistent pressure.

In some aspects, the method may involve engaging the first strap retention mechanism with a first engagement feature to retain the first strap retention mechanism in a latched position, and engaging the second strap retention mechanism with a second engagement feature to retain the second strap retention mechanism in a latched position. These engagement features may provide additional security and stability to the strap during use of the pressure application system.

### BRIEF DESCRIPTION OF THE DRAWINGS

FIG. 1 is a perspective view of an example puck in a stowed position, according to an example embodiment.

FIG. 2 is a top view of an example puck in a stowed position, according to an example embodiment.

FIG. 3 is a side view of an example puck in a stowed position, according to an example embodiment.

FIG. 4 is a front view of an example puck in a stowed position, according to an example embodiment.

FIG. 5 is a perspective view of an example puck in an orientation position, according to an example embodiment.

FIG. 6 is a top view of an example puck in an orientation position, according to an example embodiment.

FIG. 7 is a rear view of an example puck in an orientation position, according to an example embodiment.

FIG. 8 is a side cross-sectional view of an example puck in an orientation position, according to an example embodiment.

FIG. 9 is a perspective view of an example puck in an extended position, according to an example embodiment.

FIG. 10 is a top view of an example puck in an extended position, according to an example embodiment.

FIG. 11 is a rear view of an example puck in an extended position, according to an example embodiment.

FIG. 12 is a side cross-sectional view of an example puck in an extended position, according to an example embodiment.

FIG. 13 is a perspective view of an example ratchet in an extended position, according to an example embodiment.

FIG. 14 is a perspective view of an example chassis, according to an example embodiment.

FIG. 15 is a flowchart depicting a method for assembling a pressure application system. At

FIG. 16 is a flowchart depicting a method for using a pressure application system.

### DETAILED DESCRIPTION

The present disclosure is directed to a pressure application system. The pressure application system can include a harness component and a pressure puck component and be configured to provide pressure to target areas of a subject. Specifically, the present disclosure is directed to a pressure puck component (e.g., a puck) that can facilitate pre-tensioning (or tensioning) of a strap, extending of a pressure surface onto a target area of a body (e.g., axillary, brachial, subclavian, femoral, or iliac artery), and stabilizing an orientation of the pressure surface to the body and the strap. The puck may utilize a desired angle of an axis of the puck to improve the application of the pressure (e.g., to improve hemorrhage control). It is the intent of the design to advance the pressure profile and corresponding pressure surface onto the target area, while the wings stabilize the mechanism on the body.

In some embodiments, a pressure application system includes at least one pressure puck component (e.g., a puck) that is used in cooperation with at least one harness that has straps that are deployable at various locations of a body to facilitate bleeding control, tightening, pressure application, or local stabilization, among other functions. For example, the harness may be configured to be worn around a junctional area of the wearer, or proximate to the upper iliac, extremities, the aorta, or other sites of the wearer. The puck may be coupled with a strap in the harness and used to pretension the strap by the user by twisting a handle which is coupled to a fin. The twisting motion can wind the strap around a body that is coupled with or integral with the fin. Counter rotation may be prevented by a ratchet mechanism. At a threshold torque, at least one shear pin may allow decoupling of the extension means (a mechanical screw) and may advance a pressure surface into the junctional area. A wing assembly may affect and maintain stabilization of the pressure surface orientation as extension occurs. This handle may be reversibly coupled to the extension means to allow retraction of the pressure surface from the target area and allow removal or reapplication of the pressure puck component.

As an illustrative example, the pressure application system can be beneficial in combat scenarios, or other scenarios that may result in serious injuries. In such scenarios, it is important that these pucks are in close proximity to each potential subject (e.g., a warfighter) since bleed out can occur in traumatic limb hemorrhage within minutes. Therefore, having each soldier wear a harness and carry a pressure puck component may facilitate quicker response time to such injuries. Accordingly, the puck may have a stowed form factor such that the puck is easily carried. The puck may switch between a stowed form factor and a deployed form factor to effectuate proper use of the device. The pressure application system may also be applied to the subject after the injury occurs. For example, the harness may be applied after the injury occurs, rather than being worn prior to the injury.

### The Puck 100

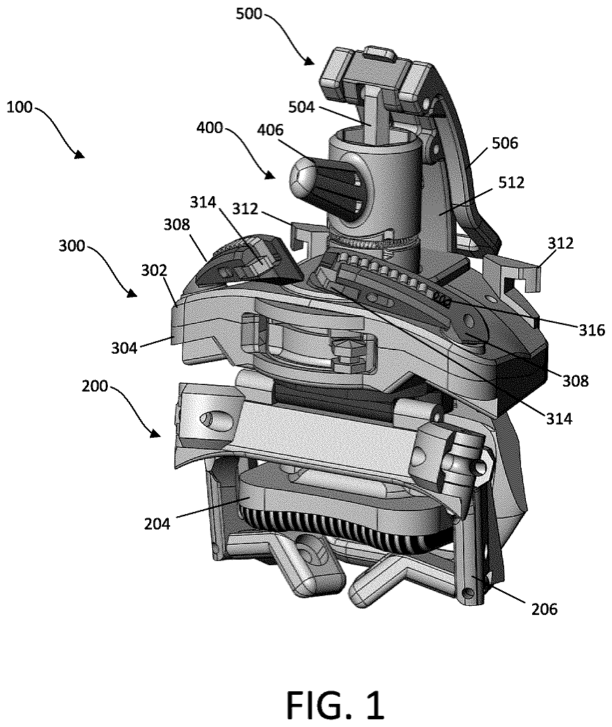

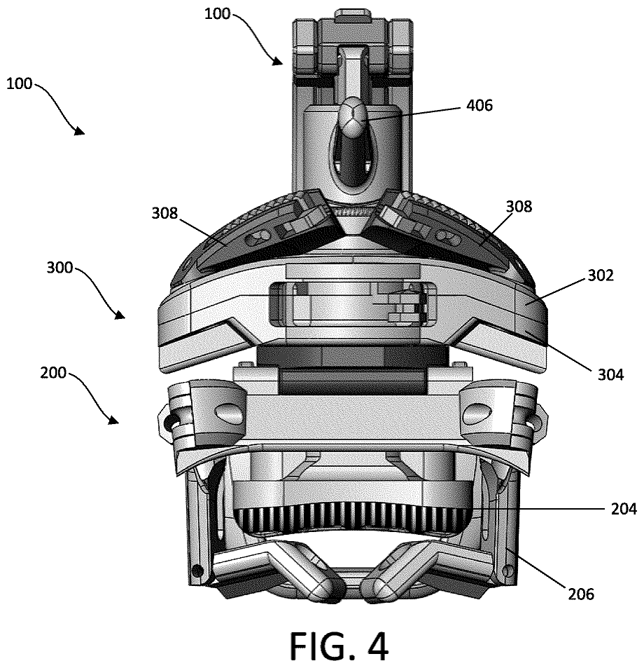

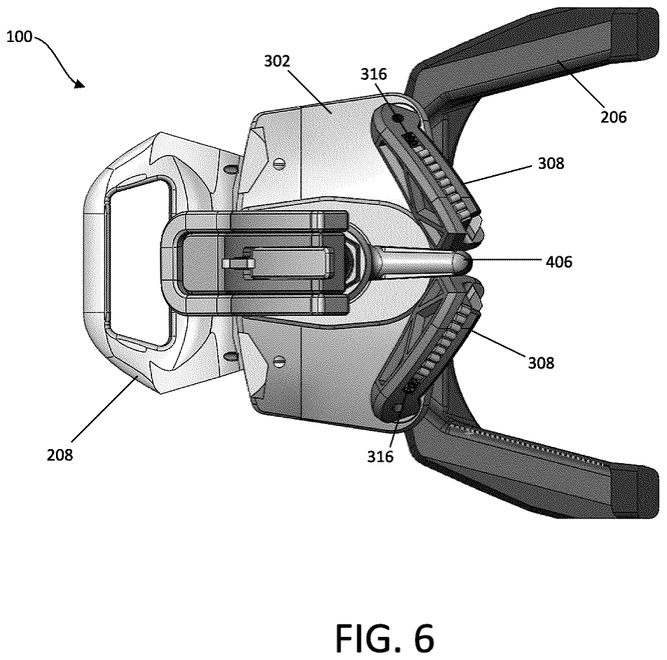

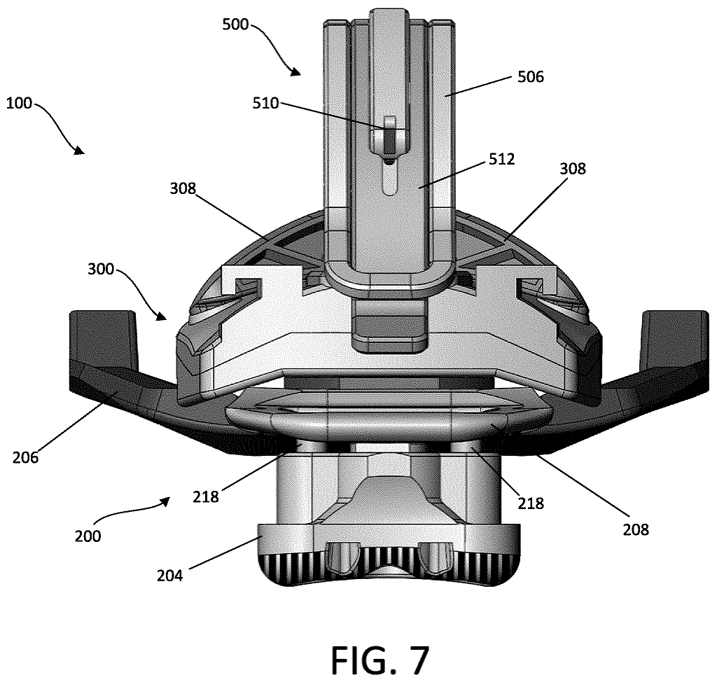

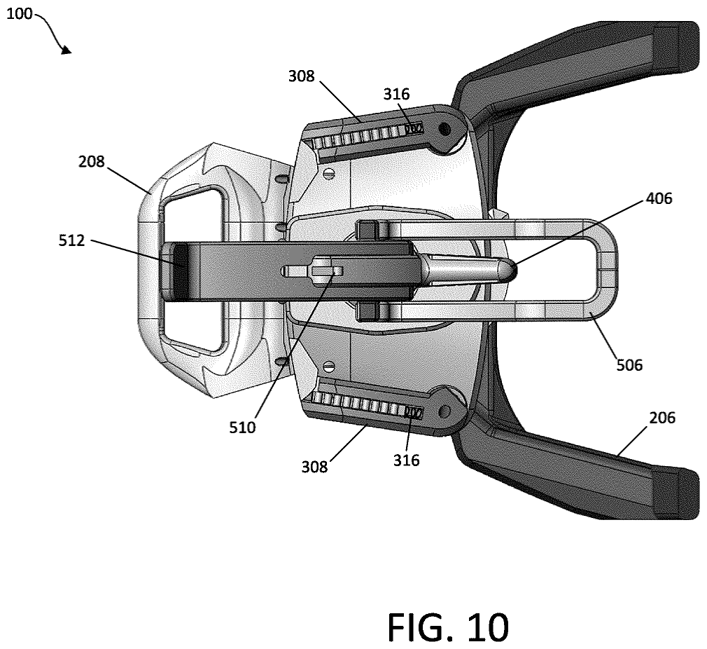

Referring to FIGS. 1-14, an example pressure puck component, shown as puck 100, is shown, according to an example embodiment. The puck 100 is a pressure application device configured to interface and engage with a strap or other component to apply pressure at or proximate a site of an injury to reduce or stop bleeding. In some embodiments, the puck 100 includes a base assembly 200. The base assembly 200 can be configured to provide stability to the puck 100 against a subject (e.g., a body of a person) to facilitate the application of the pressure to the site of the injury. The base assembly 200 can also be configured to provide the pressure to the site which may be proximal to the injury.

In some embodiments, the puck 100 includes a body, shown as chassis 300. The chassis 300 may be coupled with the base assembly 200. For example, the chassis 300 may be movably coupled with the base assembly 200 such that the base assembly 200 can move relative to the chassis 300. The chassis 300 can retain a strap at a desired position or orientation to facilitate pre-tensioning of the strap and overall tightening of the strap around the site of the injury.

In some embodiments, the puck 100 includes a strap tensioning device or extension device, shown as ratchet 400. The ratchet 400 is configured to facilitate the pre-tensioning of a strap to remove any slack in the strap when disposed around a site of an injury and engaged with the puck 100, and applying the pressure to the site of the injury. The ratchet 400 can extend through the chassis 300 and be disposed at least partially in the base assembly 200. At least a portion of the ratchet 400 can be configured to move through the chassis 300 to facilitate movement of the base assembly relative to the chassis 300.

In some embodiments, the puck 100 includes an actuator, shown as handle 500. The handle 500 can be configured to facilitate proper orientation of the strap relative to the other components of the puck 100. The handle 500 can be configured to actuate the puck 100 to initiate the pre-tensioning of the strap and the application of the pressure. The handle 500 can be manually actuated. The handle 500 can be coupled with the ratchet 400 such that movement of the handle 500 can cause movement of the ratchet 400.

### The Base Assembly 200

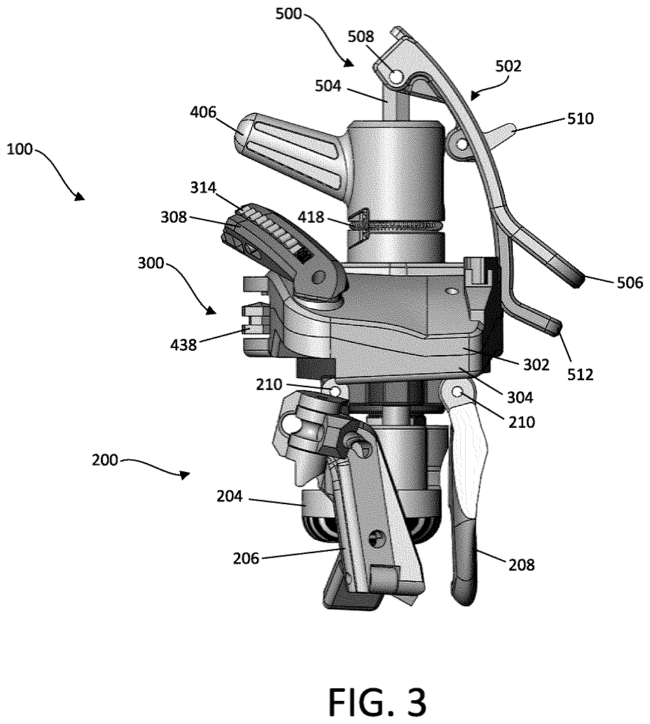

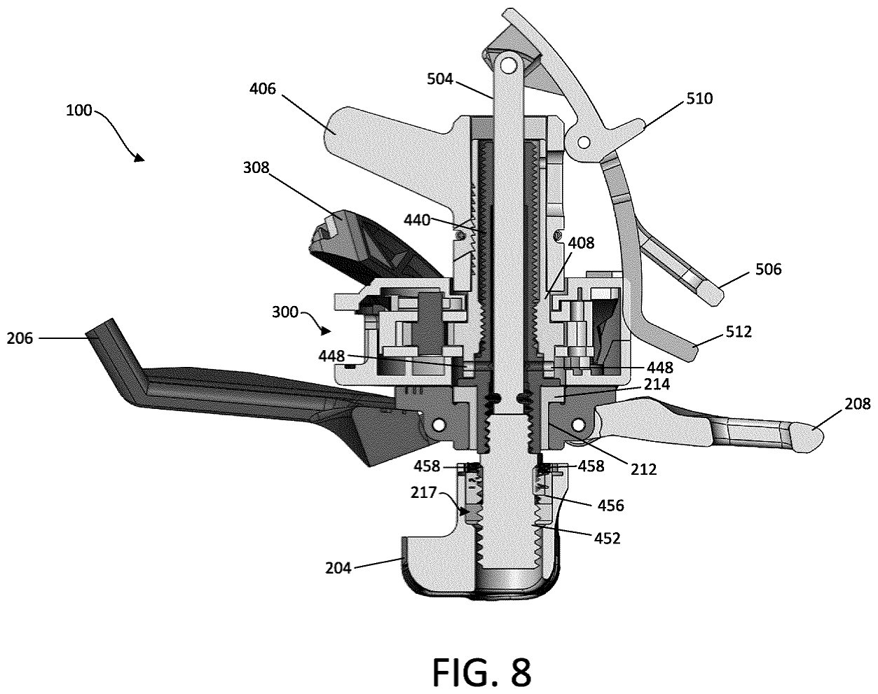

As shown in FIGS. 1, 3, 8, 9, 11, and 12, among others, in some embodiments, the base assembly 200 may include a first portion, shown as upper profile 202 and a second portion, shown as lower profile 204. The upper profile 202 can be coupled with at least one stabilizing component (e.g., a wing) to help stabilize the puck 100 against the injured subject to facilitate proper positioning of the puck 100, which can facilitate pressure being applied to the subject at a desired angle. For example, the upper profile 202 can be coupled with a first wing, shown as medial wing 206, and a second wing, shown as lateral wing 208. The wings 206, 208 can be configured to move between a first, stowed position, and a second, deployed position. For example, the wings 206, 208 can be rotatably coupled with the upper profile 202 such that the wings 206, 208 can move between the stowed position and the deployed position. In some embodiments, the wings 206, 208 may be coupled with the upper profile 202 via a hinge pin 210. The wings 206, 208 can be in the stowed position to reduce the size of the puck 100 during transport while not being used. For example, the wings 206, 208 can be rotated to extend in an axial direction (e.g., along an axial axis of the puck 100) when in the stowed position, as shown in FIG. 3, among others. The wings 206, 208 can be in the deployed position when in use. For example, the wings 206, 208 can rotate and extend laterally or radially from the upper profile 202 when in the deployed position, as shown in FIG. 8, among others. The wings 206, 208 can be configured to interface with the body of the subject to stabilize the puck 100 at a desired position.

The wings 206, 208 may be biased to the deployed position. For example, the puck 100 may be stored in a pouch when being carried and not in use. The wings 206, 208 may be biased to the deployed position such that the wings 206, 208 automatically move from the stowed position to the deployed position once removed from the pouch. In some embodiments, other retention mechanisms may keep the wings 206, 208 in the stowed position. Once the retention mechanism is removed, the wings 206, 208 may automatically move from the stowed position to the deployed position. The wings 206, 208 can be biased to the deployed position via any biasing element. For example, the biasing element may be a spring or an elastic paracord, among others.

In some embodiments, the wings 206, 208 may be static members that balance against the body of the subject. In some embodiments, the wings 206, 208 may be dynamic members. For example, the wings 206, 208 may actively deflect as the body of the subject applies pressure to the wings 206, 208. For example, the wings 206, 208 may include additional compliant members (e.g., springs), include a wire lattice (e.g., between posts of medial wings 206), or be made of a specific material. The wings may incorporate micro-mechanical structures, such as honeycomb patterns or corrugated designs, to provide both flexibility and strength. In some embodiments, the lower chassis 304 or the upper profile 202 may be configured to cause the wings 206, 208 to deflect relative to each other.

The base assembly 200 may include a base aperture 212. The base aperture 212 may extend through the upper profile 202. The base aperture 212 may be configured to receive at least a portion of the ratchet 400. For example, the ratchet 400 may extend through the base aperture 212. The base assembly 200 may include a bearing 214. In some embodiments, the bearing 214 may be a flanged thrust bearing 306. The bearing 214 may be disposed in the base aperture 212 such that the bearing 306 is encapsulated by the upper profile 202. In some embodiments, the bearing 214 may be a ball bearing. The ball bearing may provide smooth rotation between the ratchet 400 and the base assembly 200 while accommodating both radial and axial loads. In some embodiments, the bearing 214 may be a flanged ball bearing, roller bearing, a needle roller bearing, a tapered roller bearing, a plain bearing or bushing. The roller bearing may offer increased load capacity and reduced friction compared to ball bearings, which may be beneficial for applications requiring higher force transmission. Needle roller bearings may provide high radial load capacity in a compact design, which may be advantageous for space-constrained applications within the puck 100. Tapered roller bearings may be capable of handling both radial and axial loads simultaneously. Plain bearings may offer simplicity, cost-effectiveness, and the ability to operate in harsh environments. In some embodiments, the bearing 214 may be a self-lubricating bearing. Self-lubricating bearings may incorporate materials that provide lubrication during operation, potentially reducing maintenance requirements of the puck 100. In some implementations, the bearing 214 may be a ceramic bearing. Ceramic bearings may offer advantages such as lighter weight, higher hardness, and resistance to corrosion, which may enhance the durability and performance of the puck 100 in various environments. In some embodiments, the bearing 214 may be a plastic bearing. Plastic bearings may provide benefits such as lightweight construction, corrosion resistance, and the ability to operate without external lubrication. In some embodiments, the bearing 214 may be a magnetic bearing. Magnetic bearings may allow for non-contact rotation, potentially reducing wear and friction.

The lower profile 204 can be configured to apply the pressure to the subject to reduce or stop the bleeding of the subject. The lower profile 204 may have at least one pressure surface 216. The pressure surface 216 can be the surface of the lower profile 204 that contacts the subject and applies the pressure to the subject. The pressure surface 216 may also be configured to provide stability for the puck 100 to improve reliability of the application of the pressure to the subject. The lower profile 204 may define a profile pocket 217. The profile pocket 217 may be configured to receive at least a portion of a component of the puck 100 to couple the lower profile 204 with the upper profile 202.

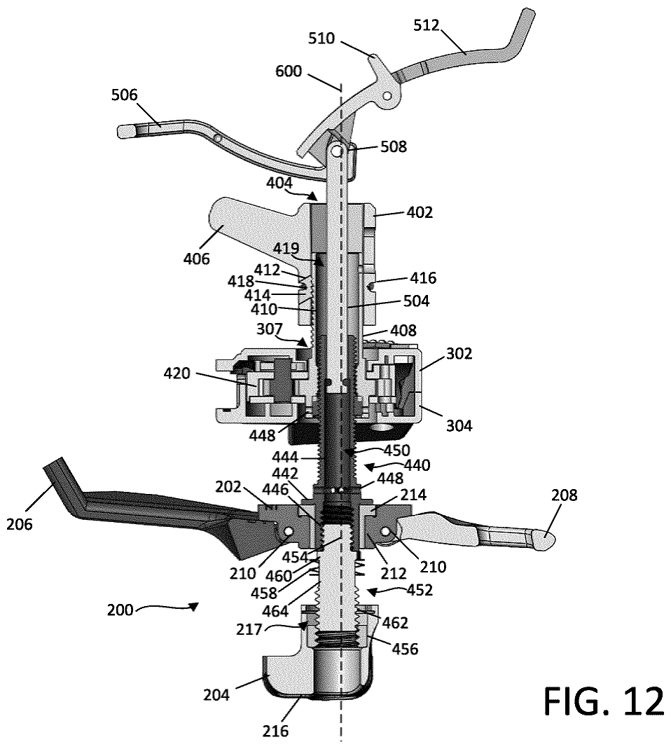

In some embodiments, the profile of the lower profile 204 may be approximately 1¼″×2⅛″ with a slight saddle like curvature. This profile may be offset as seen in FIGS. 8 and 12 to the side of the puck 100 comprising the medial wing 206. This offset may cause the strap forces to align better with reactive forces of the lower profile 204, which may lead to a smaller upsetting moment. Smaller lower profiles 204 would be less effective by missing the artery and larger lower profiles 204 may engage bone before applying sufficient occlusive pressure. The profile of the lower profile 204 may have other shapes and sizes. In some embodiments, the lower profile 204 and the wings 206, 208 may be interchangeable based on various clinical and environmental needs. For example, at least one of the lower profile 204 or the wings 206, 208 may be replaced with a different shaped component to accommodate different body shapes. In some embodiments, the lower profile 204 may include a cover configured to cover the pressure surface 216. The cover may aid in comfort and provide additional friction to increase stability. The cover may vary in size, shape, form, and material, similar to the lower profile 204.

The shape of the lower profile 204 may vary to accommodate different anatomical features and pressure application needs. In some embodiments, the lower profile 204 may have a convex shape to concentrate pressure on a specific area. In some embodiments, it may have a concave shape to distribute pressure more evenly. The lower profile 204 may also feature a tapered design, with one end being wider or thicker than the other. In some embodiments, the lower profile 204 may have an elliptical shape, an oval shape, or a rectangular shape with rounded corners. Rounded shapes provide a smoother transition of pressure application around the edges. In other implementations, the lower profile 204 may have an asymmetrical shape, with one side being longer or wider than the other to better conform to specific anatomical contours.

The curvature of the lower profile 204 may also vary. The lower profile 204 may have multiple curvatures or contours to better adapt to complex anatomical surfaces. The profile may be offset towards the side with the medial wing 206, which may improve force alignment and reduce upsetting moments.

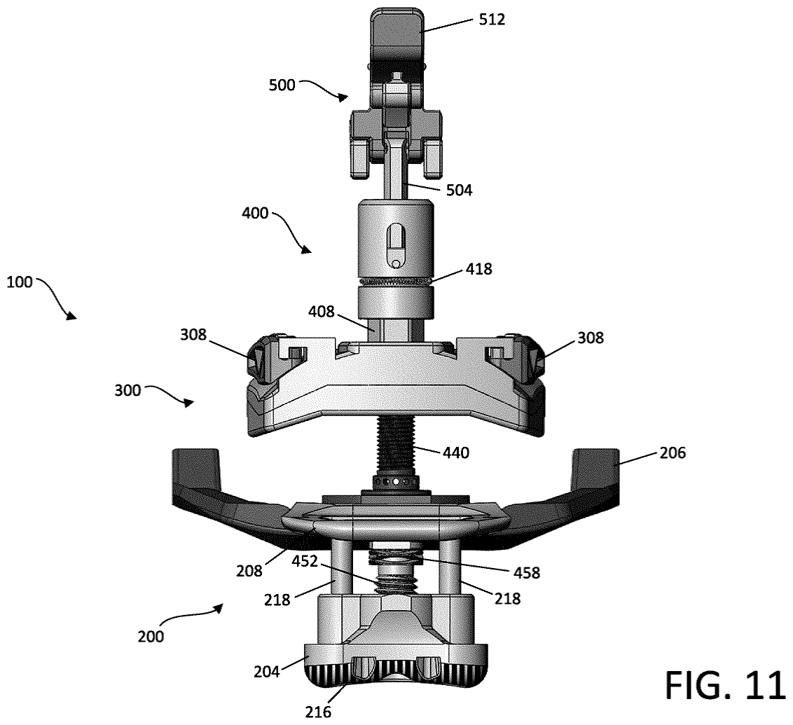

As shown in FIG. 11, among others, the base assembly 200 may include at least one extension post, shown as shoulder screw 218. The shoulder screw 218 can couple the lower profile 204 with the upper profile 202. The shoulder screw 218 can movably couple the lower profile 204 with the upper profile 202 such that the lower profile 204 can move relative to the upper profile 202. For example, the lower profile 204 may move axially relative to the upper profile 202. The lower profile 204 may move away from the upper profile 202 to increase a pressure applied to the subject via the pressure surface 216. The shoulder screw 218 may be configured to prevent rotation of the lower profile 204 relative to the upper profile 202. In some embodiments, the shoulder screw 218 may include features to prevent rotation of the lower profile 204 relative to the upper profile 202 while allowing axial movement. For example, the shoulder screw 218 may have a non-circular cross-section, such as a square or hexagonal shape, that engages with a corresponding non-circular opening in the lower profile 204. This configuration may allow the lower profile 204 to slide axially along the shoulder screw 218 while preventing rotation.

In some embodiments, the shoulder screw 218 may include a keyed feature, such as a longitudinal groove or protrusion, that mates with a corresponding feature on the lower profile 204. This keyed arrangement may allow for axial movement while restricting rotational movement.

In some embodiments, the base assembly 200 may incorporate a separate anti-rotation mechanism in addition to the shoulder screw 218. For instance, one or more guide pins may extend from the upper profile 202 and engage with linear slots or channels in the lower profile 204. These guide pins may slide within the slots, permitting axial movement while preventing rotation.

In some embodiments, the shoulder screw 218 may be replaced with or supplemented by a linear bearing system. This linear bearing system may include linear rails attached to the upper profile 202 and corresponding sliding elements on the lower profile 204. The linear bearing system may provide smooth axial movement while inherently preventing rotation.

In some embodiments, a splined shaft may be used instead of a traditional shoulder screw. The splined shaft may engage with a matching splined bore in the lower profile 204, allowing axial movement while preventing rotation due to the interlocking splines.

In some embodiments, the anti-rotation function may be achieved through the overall geometry of the base assembly 200. For example, the upper profile 202 and lower profile 204 may have complementary non-circular shapes or interlocking features that naturally prevent rotation while allowing axial movement.

The pressure application system may also utilize a combination of these approaches to ensure robust prevention of rotation between the lower profile 204 and the upper profile 202 while maintaining the desired axial movement capability.

In some embodiments, the base assembly 200 may include a plurality of shoulder screws 218. For example, the base assembly 200 may include a first shoulder screw 218 and a second shoulder screw 218. The first shoulder screw 218 may be disposed proximate to a first side of the lower profile 204 and the second shoulder screw 218 may be disposed proximate to a second side of the lower profile 204.

### The Chassis 300

As shown in FIGS. 1, 5, and 8, among others, in some embodiments, the chassis 300 includes an upper chassis 302 and a lower chassis 304. The upper chassis 302 and the lower chassis 304 can be coupled together to form a single housing. In some embodiments, the upper chassis 302 is coupled with the lower chassis 304 via a plurality of mechanical fasteners (e.g., screws). The chassis 300 can be coupled with the base assembly 200. For example, the chassis 300 can be movably coupled with the base assembly 200 via the ratchet 400 such that the base assembly 200 can move relative to the chassis 300. The upper profile 202 of the base assembly 200 can be disposed between the chassis 300 and the lower profile 204 of the base assembly 200.

The chassis 300 can include at least one chassis aperture 307. The chassis aperture 307 can extend through both the upper chassis 302 and the lower chassis 304. The chassis aperture 307 can be configured to receive at least a portion of the ratchet 400. For example, a portion of the ratchet 400 may be disposed in the chassis aperture 307. The portion of the ratchet 400 may couple the ratchet 400 with the chassis 300.

In some embodiments, the chassis 300 includes at least one strap retention mechanism 308, shown as a strap stay in FIG. 3. In some embodiments, the chassis 300 may include alternative strap retention mechanisms to the strap stay. These alternative mechanisms may include:
A cam cleat mechanism that may allow quick and secure strap retention with the ability to adjust tension easily. The cam cleat may include spring-loaded cams that grip the strap when tension is applied;
A friction buckle system that may use a series of bars or rollers to create a serpentine path for the strap, increasing friction and holding the strap in place;
A ratchet buckle mechanism that may allow incremental tightening and easy release of the strap. This mechanism may include a gear and pawl system for precise adjustments;
A magnetic clasp system that may use strong magnets to secure the strap in place. This system may allow for quick attachment and detachment while providing secure retention;
A hook-and-loop fastener system that may offer adjustable and secure strap retention. This system may allow for easy repositioning and adjustment of the strap;
A spring-loaded clamp mechanism that may automatically grip the strap when inserted and release when a lever is actuated. This mechanism may provide quick and secure strap retention;
A sliding lock mechanism that may use a spring-loaded slider to pinch the strap against a fixed surface, securing it in place. This mechanism may allow for smooth adjustment and quick release;
An eccentric cam mechanism that may use a rotating cam to increase friction on the strap as tension is applied. This mechanism may provide variable retention force based on strap tension;
A serrated jaw mechanism that may use opposing serrated surfaces to grip the strap. This mechanism may be actuated by a lever or screw to increase or decrease gripping force; or
A rotary dial system that may allow the user to wind excess strap material around a spool, securing it in place. This system may provide both retention and storage for excess strap length.

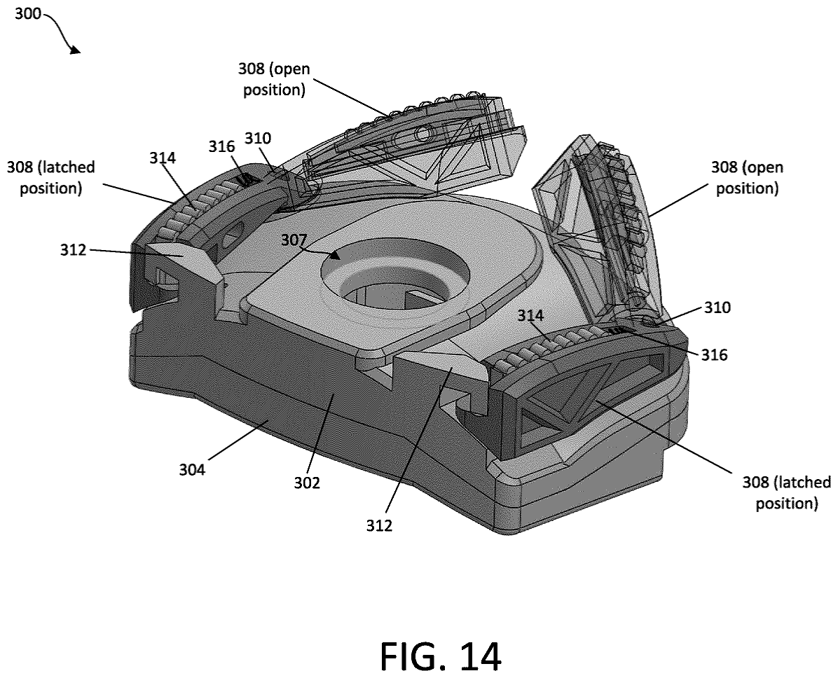

The strap retention mechanism 308 can be configured to retain a portion of the strap and keep the portion of the strap at a certain position or orientation to facilitate tensioning of the strap. The strap retention mechanism 308 may be coupled with the chassis 300. In some embodiments, the strap retention mechanism 308 is coupled with the upper chassis 302 of the chassis 300. As shown in FIG. 14, among others, the strap retention mechanism 308 may be movably coupled (e.g., rotatably coupled) with the chassis 300. For example, the strap retention mechanism 308 may be coupled with the chassis 300 at a pivot point 310, and may be configured to rotate around the pivot point 310 to move between an open position and a latched position. With the strap retention mechanism 308 in the open position, the strap can be positioned at a desired location relative to the chassis 300. With the strap in the desired position, the strap retention mechanism 308 can move to the latched position to maintain the position of the strap.

In some embodiments, the strap retention mechanism 308 may be rotatably coupled to the chassis using alternative mechanisms. For example:

The strap retention mechanism 308 may be coupled to the chassis using a living hinge, which may be integrally formed with the strap retention mechanism 308 and chassis as a single piece of flexible material. This configuration may reduce part count and simplify assembly.

A ball and socket joint may be used to couple the strap retention mechanism 308 to the chassis, allowing for multi-directional movement in addition to rotation. This may provide additional flexibility in positioning the strap.

The strap retention mechanism 308 may be coupled to the chassis using a pin-in-slot mechanism, where a pin on the strap retention mechanism 308 slides within a curved slot on the chassis. This may allow for both rotational and slight translational movement.

A flexible coupling, such as a rubber or elastomeric joint, may be used to connect the strap retention mechanism 308 to the chassis. This may provide both rotational movement and some shock absorption.

The strap retention mechanism 308 may be coupled to the chassis using a magnetic pivot, where opposing magnets in the strap retention mechanism 308 and chassis allow for rotation while also providing a holding force in the closed position.

A ratcheting mechanism may be incorporated into the rotatable coupling, allowing the strap retention mechanism 308 to be locked at various angles between the open and closed positions.

The strap retention mechanism 308 may be coupled to the chassis using a spring-loaded hinge mechanism that biases the strap retention mechanism 308 towards either the open or closed position, depending on the desired functionality.

The chassis 300 may include at least one engagement feature, shown as keeper 312. The keeper 312 may be configured to engage with the strap retention mechanism 308 to keep the strap retention mechanism 308 in the latched position. For example, the strap retention mechanism 308 may include a latching mechanism, shown as latch 314. The latch 314 may be coupled with the strap retention mechanism 308 via at least one mechanical fastener (e.g., a pin).

The latch 314 may be coupled with the strap retention mechanism 308 via various alternative methods, including:
Integral molding: The latch may be integrally molded with the strap retention mechanism as a single piece, eliminating the need for separate fasteners;
Snap-fit connection: The latch may incorporate snap features that securely engage with corresponding receptacles on the strap retention mechanism;
Adhesive bonding: A strong adhesive may be used to bond the latch to the strap retention mechanism, providing a permanent connection;
Welding: For certain materials, the latch may be welded to the strap retention mechanism using techniques such as ultrasonic welding or heat staking;
Magnetic coupling: The latch and strap retention mechanism may incorporate embedded magnets for a secure yet detachable connection;
Dovetail joint: A dovetail-shaped protrusion on the latch may slide into a corresponding groove on the strap retention mechanism, creating a secure mechanical interlock;
Press-fit assembly: The latch may be designed with tight tolerances to press-fit into a receiving feature on the strap retention mechanism;
Threaded connection: The latch may include threaded features that screw into corresponding threaded holes in the strap retention mechanism;
Riveting: The latch may be permanently attached to the strap retention mechanism using rivets; or
Hook-and-loop fastener: For applications requiring adjustability, hook-and-loop fasteners may be used to attach the latch to the strap retention mechanism.

The latch 314 may be configured to move relative to the strap retention mechanism 308. For example, the strap retention mechanism 308 may include a compliant member, shown as latch spring 316. The latch spring 316 may be configured to bias the latch 314 to a first position, and facilitate movement of the latch between the first position and a second position to facilitate engagement of the latch 314 with the keeper 312. The keeper 312 may retain the strap retention mechanism 308 in the latched position by engaging with the latch 314. The strap retention mechanism 308 may have any configuration configured to hold the strap in place. For example, the latch 314 may be a rope or string-like element configured to be received by the keeper 312 to maintain a position of the strap retention mechanism 308.

In some embodiments, the chassis 300 may include a plurality of strap retention mechanisms 308. For example, the chassis 300 may include a first (e.g., a left) strap retention mechanism 308 and a second (e.g., a right) strap retention mechanism 308. The first strap retention mechanism 308 may be disposed on a first side of the chassis 300 and the second strap retention mechanism 308 may be disposed on a second side of the chassis 300. For example, the first strap retention mechanism 308 may be disposed on a first side of the chassis aperture 307 and the second strap retention mechanism 308 may be disposed on a second side of the chassis aperture 307. Each strap retention mechanism 308 may have a latch 314. The chassis 300 may include a plurality of keepers 312 to keep the plurality of strap retention mechanisms 308 in latched positions. For example, the chassis 300 may include a first keeper 312 to engage with the latch 314 of the first strap retention mechanism 308 and a second keeper 312 to engage with the latch 314 of the second strap retention mechanism 308.

### The Ratchet 400

As shown in FIGS. 1, 8, 12, and 13, among others, the ratchet 400 may be configured to couple with the various components of the puck 100 to couple the various components together and to drive the components to facilitate tensioning of the strap and applying pressure to the subject. In some embodiments, the ratchet 400 may include a body 402. The body 402 can be disposed on a first side of the chassis 300. For example, the body 402 can be disposed adjacent to the upper chassis 302. The chassis 300 can be disposed between the body 402 and the upper profile 202 of the base assembly 200. The body 402 can be movably coupled with the chassis 300. For example, the chassis 300 can move relative to the body 402. The body 402 may define a body aperture 404 configured to receive another component of the ratchet 400. The body aperture 404 may extend axially through at least a portion of the body 402. In some embodiments, the body aperture 404 may extend through the entire body 402. The body aperture 404 may have a geometric shape. In some embodiments, the body aperture 404 may have a hexagonal shape. The shape of the body aperture 404 may be based on a shape of a component of the ratchet 400 configured to be disposed in the body aperture 404. In some embodiments, the body aperture 404 may have other geometric shapes to accommodate different components of the ratchet 400, such as: square, which may provide a simple and secure engagement with a corresponding square component; octagonal, offering more points of contact than a hexagonal shape for potentially improved torque transfer; splined, which may allow for precise alignment and efficient torque transmission; D-shaped, combining a flat side with a curved side for a unique keying feature; triangular, which may provide a compact design with three points of contact; star or Torx-like shape, offering multiple points of engagement for high torque applications; oval or elliptical, which may accommodate components that require orientation in a specific direction; a keyed circular shape, featuring a circular aperture with one or more keyways for alignment purposes; a polygonal shape with an odd number of sides (e.g., pentagonal, heptagonal), which may provide unique engagement properties; or a lobed shape, such as a tri-lobe or quad-lobe profile, which may offer a combination of smooth rotation and positive engagement.

The ratchet 400 may include a projection, shown as fin 406. The fin 406 may extend from a side of the body 402. The fin 406 may be configured to hold or retain a portion of the strap. For example, the strap may be configured to rest on a top surface of the fin 406. The fin 406 may extend in an upward direction from the side of the body 402 to prevent the strap from sliding off of the fin 406. The fin 406 may be disposed proximate to a top of the body 402 (e.g., away from the chassis 300. The fin 406 may be disposed a predetermined distance from the chassis 300 to facilitate proper tensioning of the strap.

In some embodiments, the ratchet 400 includes a spool 408. The spool 408 can be movably coupled with the body 402. For example, the spool 408 can be disposed at least partially in the body aperture 404. The spool 408 may be configured to slide within the body aperture 404 in an axial direction. The spool 408 may have a shape that corresponds with the shape of the body aperture 404. For example, the spool 408 may have a hexagonal shape to match a hexagonal shape of the body aperture 404. The shape of the body aperture 404 and the spool 408 may be configured to prevent rotation of the spool 408 relative to the body 402. The spool 408 may include at least one spool groove 410 configured to prevent downward motion of the fin 406 and the body 402 when the strap provides an axial load on the puck 100.

In some embodiments, the body 402 may include at least one lock groove 412. A lock 414 may be disposed in the lock groove 412. The lock 414 may be configured to engage with at least one spool groove 410 to prevent the downward motion of the body 402 and the fin 406. The ratchet 400 may include a spring groove 416 that extends around body 402 and around the lock 414. For example, a first portion of the spring groove 416 extends into the body 402 and a second portion of the spring groove 416 extends into the lock 414. The first portion and the second portion align with the lock 414 in the lock groove 412 to create a continuous spring groove 416. A compliant member, shown as garter spring 418 can be disposed in the spring groove 416. The garter spring 418 can keep the lock 414 in contact with the spool groove 410 to maintain the axial locking under the load from the strap. The garter spring 418 can prevent the downward motion of the body 402 relative to the spool 408, but may also allow upward motion of the body 402 relative to the spool 408, such that the spool 408 can slide out of the body 402.

In some embodiments, the compliant member may be a coil spring, a wave spring, a Belleville washer, or a stack of Belleville washers; an elastomeric ring; a leaf spring; a torsion spring, a compression spring, a conical spring, a polymer spring, or a magnetic spring.

In some embodiments, the body 402 may include at least one slot 415. The slot 415 may extend through a wall of the body 402. The ratchet 400 may include at least one spool retention member, shown as retention pin 417. The retention pin 417 may be coupled with or be integral with the spool 408 and be disposed at least partially in the slot 415. The retention pin 417 may be configured to translate within the slot 415 to retain a rotational position of the fin 406 and facilitate axial movement of the body 402. In some embodiments, the retention pin 417 may be a screw. In some embodiments, the retention pin 417 may be: a press-fit pin, a cylindrical pin with tight tolerances that may be press-fit into the spool 408 and extend into the slot 415; a snap-fit pin, a pin with a flexible head may snap into a corresponding hole in the spool 408 and protrude into the slot 415; a riveted pin: a pin permanently attached to the spool 408 through a riveting process; a welded pin that is welded or brazed to the spool 408; an integrally molded pin; a magnetic pin that is attracted to a ferrous insert in the spool 408; a spring-loaded pin inserted into the spool 408 and extending into the slot 415 under spring tension; a Cotter pin; a threaded insert with setscrew, allowing for adjustment to the desired protrusion into the slot 415; and a dovetail pin, sliding into a corresponding dovetail slot in the spool 408, providing both retention and resistance to rotation.

The spool 408 may define a spool aperture 419. The spool aperture 419 may extend axially along the spool 408. In some embodiments, the spool aperture 419 extends through the entire spool 408. The spool aperture 419 may be configured to receive other components of the puck 100.

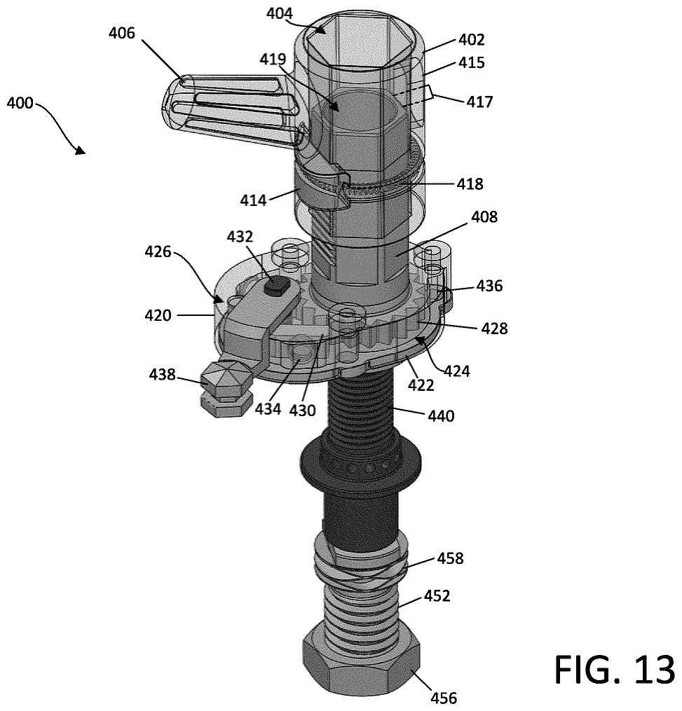

In some embodiments, the ratchet 400 includes at least one case 420. The case 420 can be coupled with the spool 408. The case 420 can be disposed in the chassis 300. The spool 408 may be coupled with the chassis 300 via the case 420. The ratchet 400 may include a plate 422. The case 420 and the plate 422 can define a cavity 424. The case 420 and the plate 422 can be configured to house a rotation control mechanism, shown as ratchet mechanism 426. The ratchet mechanism 426 can be configured to control rotation of the spool 408 and the fin 406. For example, the spool 408 may include radial ratchet teeth 428 that are disposed in the cavity 424. The radial ratchet teeth 428 may be configured to engage with the ratchet mechanism 426 to prevent anti-rotation of the spool 408 and the fin 406. As shown in FIG. 13, among others, in some embodiments, the ratchet mechanism 426 may include at least one pawl 430, at least one cam 432, at least one ratchet spring 434, at least one screw 436, and at least one lever 438. The screw 436 may be configured to couple the plate 422 with the case 420. The lever 438 may be configured to release the body 402 and the fin 406 such that the strap can become loose. In some embodiments, the ratchet mechanism 426 may include a plurality of leaf springs instead of or in addition to the pawl 430.

In some embodiments, the ratchet mechanism 426 may include alternative components instead of, or in addition to, the pawl 430 and leaf springs. These alternatives may include:
A roller clutch: A roller clutch may provide smooth, silent operation and allow for instant engagement in one direction while allowing free rotation in the opposite direction;
A sprag clutch: Similar to a roller clutch, a sprag clutch may offer high torque capacity in a compact design, potentially suitable for applications requiring higher load-bearing capabilities;
A ratchet and pawl with multiple engagement points: This design may use multiple pawls or a pawl with multiple teeth to increase the number of engagement points, potentially providing finer control and smoother operation;
A friction disc system: A stack of friction discs may be used to create resistance in one direction while allowing free rotation in the other, potentially offering adjustable tension;
A centrifugal clutch mechanism: This system may engage automatically as rotational speed increases, potentially providing a speed-dependent locking mechanism;
A magnetic detent system: A series of magnets may be arranged to create preferential stopping points, potentially offering a non-contact alternative to mechanical engagement;
A Geneva mechanism: This may provide intermittent rotary motion, potentially useful for applications requiring precise incremental movements;
An escapement mechanism: Similar to those used in clockwork, an escapement mechanism may offer precise control over rotational movement;
A wrap spring clutch: This may provide instant engagement and disengagement, potentially useful for rapid response applications; and
A ball detent mechanism: A spring-loaded ball bearing may engage with notches or depressions, potentially offering a simple, low-profile alternative to traditional pawl systems.

In some embodiments, the ratchet 400 includes at least one primary screw 440. The primary screw 440 can be configured to facilitate tensioning of the strap as well as extending the puck 100 such that the lower profile 204 can apply pressure to the subject. The primary screw 440 can have a primary flange 442. The primary flange 442 can extend radially away from the primary screw 440. The primary flange 442 can divide the primary screw 440 into a first portion 444 and a second portion 446. The first portion 444 can be configured to be at least partially disposed in the spool aperture 419. The first portion 444 may be configured to couple the primary screw 440 with the spool 408. For example, the first portion 444 may be threadedly coupled with the spool 408.

In some embodiments, the first portion 444 may include a shear pin 448. The shear pin 448 may extend between the first portion 444 of the primary screw 440 and the spool 408. The shear pin 448 may be configured to prevent rotation of the primary screw 440 relative to the spool 408 until a force applied to the shear pin 448 reaches a torque threshold. At the torque threshold, the shear pin 448 may break and allow the primary screw 440 to rotate relative to the spool 408. The shear pin 448 may be designed to accommodate different torque thresholds. For example, the size, shape, or material of the shear pin 448 may be adjusted or modified to achieve a different torque threshold. The diameter of the shear pin could be varied to alter its shear strength. The length of the shear pin could be adjusted to change the shear plane. The shear pin could be fabricated from different metals or alloys with varying yield strengths, such as steel, aluminum, brass, or titanium alloys. Composite materials like carbon fiber-reinforced polymers could also be used to create shear pins with specific failure characteristics.

The cross-sectional shape of the shear pin could be modified, for instance using circular, square, hexagonal, or custom profiles to influence shear behavior. Multiple smaller shear pins could be used instead of a single larger pin. The shear pin could incorporate intentional stress concentration features like notches or holes to create predetermined failure points. Heat treatment or surface hardening processes could be applied to the shear pin to alter its mechanical properties. The interface between the shear pin and surrounding components could be designed with tight or loose tolerances to affect load distribution.

Additionally, the shear pin could be replaced with alternative torque-limiting mechanisms like clutch systems, detent mechanisms, or torsion springs calibrated to disengage at specific torque levels. Electronically-controlled actuators can be employed to provide adjustable torque thresholds. These various design options allow the torque threshold to be precisely tuned for different applications or user requirements. With the threadable coupling between the first portion 444 of the primary screw 440 and the spool 408, rotation of the primary screw 440 relative to the spool 408 may cause the primary screw 440 to translate in an axial direction relative to the spool 408 such that a section of the first portion 444 is disposed outside of the spool 408 (e.g., below the spool 408).

The second portion 446 may be configured to couple the primary screw 440 with the base assembly 200. For example, the second portion 446 may be disposed in the base aperture 212. The second portion 446 may be disposed in the bearing 214 such that the bearing 214 is disposed between the second portion 446 and the upper profile 202.

The primary screw 440 may define a screw aperture 450. The screw aperture 450 may extend axially along the primary screw 440. The screw aperture 450 may extend the full length of the primary screw 440. For example, the screw aperture 450 may extend through the first portion 444 and the second portion 446. A first section of the screw aperture 450 may have a polygonal shape. The shape of the first section of the screw aperture 450 may be based on a shape of a corresponding component of the puck 100. The first section may be a part of the first portion 444. For example, the first section may be disposed in the spool 408. A second section of the screw aperture 450 may have a different shape than the first section. The second shape may be based on a shape of a different corresponding component of the puck 100.

The ratchet 400 may include at least one secondary screw 452. The secondary screw 452 can be coupled with the primary screw 440. For example, the secondary screw 452 can be fixedly coupled with the primary screw 440 such that rotation of the primary screw 440 can cause rotation of the secondary screw 452. The secondary screw 452 may be at least partially disposed between the upper profile 202 and the lower profile 204 of the base assembly 200. The secondary screw 452 may be coupled with the second portion 446 of the primary screw 440. For example, the secondary screw 452 may include a head 454. The head 454 may be disposed in the second section of the screw aperture 450 that is disposed in the second portion 446 of the primary screw 440. The head 454 can couple the secondary screw 452 with the primary screw 440.

With at least one shoulder screw 218 preventing rotation of the lower profile 204 relative to the upper profile 202, rotation of the secondary screw 452 may cause the lower profile 204 to extend axially away from the upper profile 202. For example, the ratchet 400 may include at least one nut 456. The nut 456 may be disposed in the profile pocket 217 of the lower profile 204. The nut 456 may be rigidly coupled with the lower profile 204 such that the nut 456 does not rotate within the profile pocket 217.

In some embodiments, the ratchet 400 may include at least one spring 458, shown in the figures as a wave spring. In other embodiments, not shown, the spring 458 may be provided as: a stack of Belleville washers, offering adjustable spring force and a more compact axial profile; a coil spring, potentially providing more linear force characteristics; a conical spring could be employed to offer variable force characteristics as it compresses; a leaf spring configuration potentially offering a flatter profile and customizable force distribution; a gas spring, allowing for adjustable pressure and smoother operation; an elastomeric component could serve as a spring, offering vibration damping properties in addition to spring force; a system of repelling magnets could create a spring-like effect without traditional mechanical components; a spring made from composite materials like carbon fiber could offer high strength-to-weight ratio and corrosion resistance; a spring made from shape memory alloys could provide unique force characteristics and the ability to change properties with temperature; and a pneumatic system could be used to create spring-like behavior, allowing for adjustable force and potential energy storage.

The spring 458 can be disposed between the nut 456 and a secondary flange 460 of the secondary screw 452. The spring 458 can be configured to facilitate reengagement. For example, the secondary screw 452 may have a lower threaded portion 462 configured to couple with the nut 456. The secondary screw 452 may have a cylindrical portion 464 above the lower threaded portion 462 of the secondary screw 452. The cylindrical portion 464 may be smaller than an inner diameter of the nut 456. With the nut 456 on the cylindrical portion 464, the primary screw 440 may continue to unscrew. The spring 458 may be configured to reengage the nut 456 onto the threaded portion 462 of the secondary screw 452 when rotating in the direction that extends the primary screw 440. For example, the spring 458 may be configured to transition nut 456 and secondary screw 452 from rotatably and slidably coupled to screwably coupled when the primary screw 440 is rotated in the direction that extends the primary screw 440 relative to the spool 408.

The ratchet 400 described herein is an example of a core assembly of the puck 100. The puck 100 may include other variations of the ratchet 400. For example, the ratchet 400 may include any type of ratchet mechanism 426 to prevent anti-rotation of the fin 406. In some embodiments, instead of the double screw mechanism (e.g., the primary screw 440 and the secondary screw 452), the ratchet 400 may include a gas spring. In some embodiments, the shear pins 448 may be a part of a reloadable cartridge such that the puck 100 may be used any number of times. In some embodiments, the shear pins 448 may be non-destructive such that the same shear pins may be reusable. In some embodiments, the puck 100 may include specific heat treated screws and nuts for better performance and reliability.

### The Handle 500

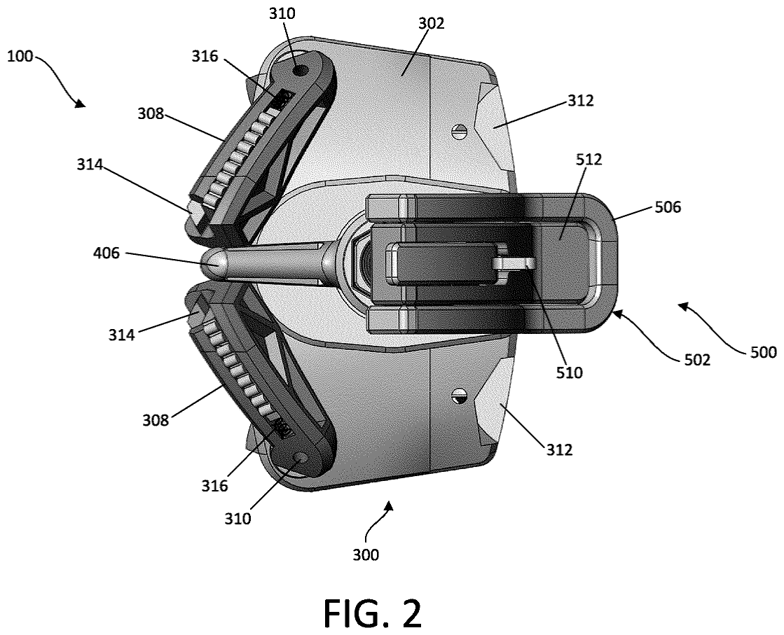

As shown in FIGS. 2, 3, 9, and 12, among others, in some embodiments, the handle 500 may include a handle portion 502 and a drive post 504. The handle portion 502 may be coupled with the drive post 504. The handle portion 502 may be coupled at an end of the drive post 504. The handle portion 502 may be configured to drive or actuate the drive post 504. For example, a user may grab or hold the handle portion 502 and twist or rotate the handle portion 502 in order to rotate the drive post 504.

The handle portion 502 may include at least one handle. For example, the handle portion 502 may include an outer handle 506. The outer handle 506 may be coupled to the drive post 504. In some embodiments, the outer handle 506 may be rotatably coupled with the drive post 504. For example, the outer handle 506 may be coupled with the drive post 504 via a handle hinge pin 508. The outer handle 506 may be configured to rotate around the handle hinge pin 508 to move between a receiving position and a first rotation position. In the receiving position, the outer handle 506 may extend axially along the ratchet 400 such that an end of the outer handle 506 is disposed adjacent to the chassis 300. In the receiving position, a strap may disposed on the handle portion 502. The handle portion 502 may include a retention feature, shown as catch 510. The catch 510 may be or include a projection to hold the strap at a desired location to facilitate proper placement of the strap for tensioning. With the strap disposed on the handle portion 502, the outer handle 506 may be configured to rotate about the handle hinge pin 508 to the first rotation position. With the outer handle 506 in the first rotation position, the strap may release from the handle portion 502 and drop onto the fin 406 of the rachet 400. The outer handle 506 may define a outer handle aperture 511.

In some embodiments, the handle portion 502 may include an inner handle 512. The inner handle 512 may be coupled with the drive post 504. In some embodiments, the inner handle 512 may be coupled with the drive post 504 via the handle hinge pin 508. The inner handle 512 may be configured to move between a receiving position and a second rotation position. For example, in the receiving position, at least a portion of the inner handle 512 may be disposed in the outer handle aperture 511 and extend axially along the ratchet 400 such that an end of the inner handle 512 is disposed adjacent to the chassis 300. The inner handle 512 may be longer than the outer handle 506. The inner handle 512 may rotate about the same handle hinge pin 508 to the second rotation position. The outer handle 506 may rotate further around the handle hinge pin 508 than the inner handle 512 such that the first rotation position may include the outer handle 506 being disposed on a first side of the drive post 504 and the second rotation position may include the inner handle 512 being disposed on a second side of the drive post 504. The outer handle 506 and the inner handle 512 extending in different directions from the drive post 504 may provide a handle 500 that is easy for a user to grab and apply the necessary force to tension the strap and apply pressure to the site of the injury.

The inner handle 512 may define an inner handle aperture 514. At least a portion of the catch 510 may be disposed in the inner handle aperture 514. The catch 510 may be configured to move with the inner handle 512.

The drive post 504 may be configured to extend into the ratchet 400 of the puck 100. For example, the drive post 504 may extend through the body 402 via the body aperture 404. The drive post 504 may extend into or through the spool 408 via the spool aperture 419. The drive post 504 may extend into the primary screw 440 via the screw aperture 450. The drive post 504 may be configured to couple the handle 500 with the primary screw 440. For example, the drive post 504 may interface with and engage with the first portion 444 of the primary screw 440. A shape of the drive post 504 may be configured to engage with the primary screw 440 such that rotation of the drive post 504 can cause rotation of the secondary screw 452. For example, the shape of a first section of the screw aperture 450 may be hexagonal. The shape of the drive post 504 may be hexagonal. Some alternative configurations for the screw aperture 450 and drive post 504 include: a splined profile, allowing for multiple points of contact and potentially higher torque capacity; a square cross-section for both components, which can provide a simple yet effective engagement mechanism; a triangular profile, offering a compact design with three points of contact; an octagonal cross-section, providing more points of contact than a hexagonal shape, potentially improving torque transfer and reducing wear; a star-shaped or Torx-like profile could offer multiple points of engagement, potentially reducing the risk of stripping under high torque loads; a circular cross-section with one or more keyways to provide both rotational engagement and axial alignment; an oval or elliptical profile could offer orientation-specific engagement, ensuring proper alignment during assembly; a polygonal shape with an odd number of sides, such as pentagonal, heptagonal, or nonagonal, could provide unique engagement properties and potentially reduce manufacturing tolerances; the drive post 504 and screw aperture 450 could have slightly tapered profiles to ensure a snug fit and reduce play in the mechanism; and an asymmetrical profile could be designed to ensure the drive post 504 can only be inserted into the screw aperture 450 in the correct orientation.

### Extension and Retraction of the Puck 100

The puck 100 may be configured to move between a stowed position and an extended position, with various intermediate positions in between. FIGS. 1-4 show the puck 100 in a stowed position, in accordance with an example embodiments. In the stowed position, the outer handle 506 and the inner handle 512 may be disposed in the receiving position. For example, the outer handle 506 and the inner handle 512 may extend axially along the puck 100 such that ends of the handles 506, 512 are disposed adjacent to the chassis 300. In the stowed position, the strap retention mechanism 308 may be in an open position and the wings 206, 208 may be in a stowed position. In the stowed position, the puck 100 may be in a fully retracted state such that the spool 408 may be fully retracted into the body 402, the primary screw 440 may be fully retracted into the spool 408, and the lower profile 204 may be full retracted over the secondary screw 452. The stowed position of the puck 100 reduces the form factor of the puck 100 to make the puck 100 easier to carry.

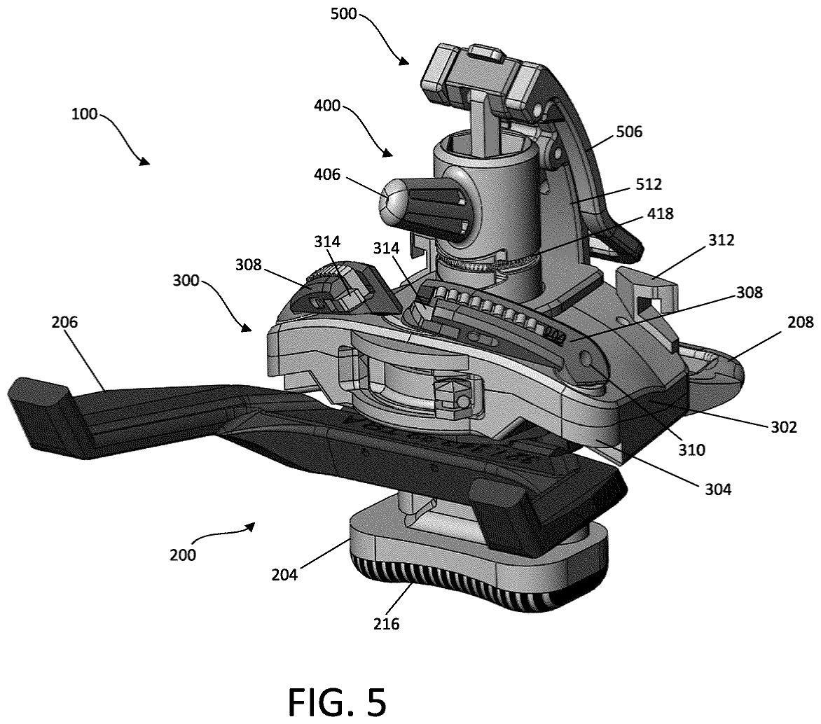

As shown in FIGS. 5-8, among others, once a retention mechanism is removed from the puck 100 (e.g., the puck 100 is removed from a pouch), the puck 100 may transition from the stowed position to an orientation position. In the orientation position, the wings 206, 208 may automatically move from the stowed position to the deployed position. In the deployed position, the wings 206, 208 extend radially from the upper profile 202. The orientation position of the puck 100 facilitates proper placement of the puck 100 on the subject to facilitate proper application of pressure (e.g., location and orientation) to the subject.

As an example, in the orientation position, the medial wing 206 and lateral wing 208 may be positioned so that the puck 100 may be positioned on the body of the subject. The lower profile 204 may be placed in close contact with, for example, an artery of the subject. The medial wing 206 may wrap around the breast of the subject and the lateral wing 208 may contact the upper arm and shoulder of the subject.

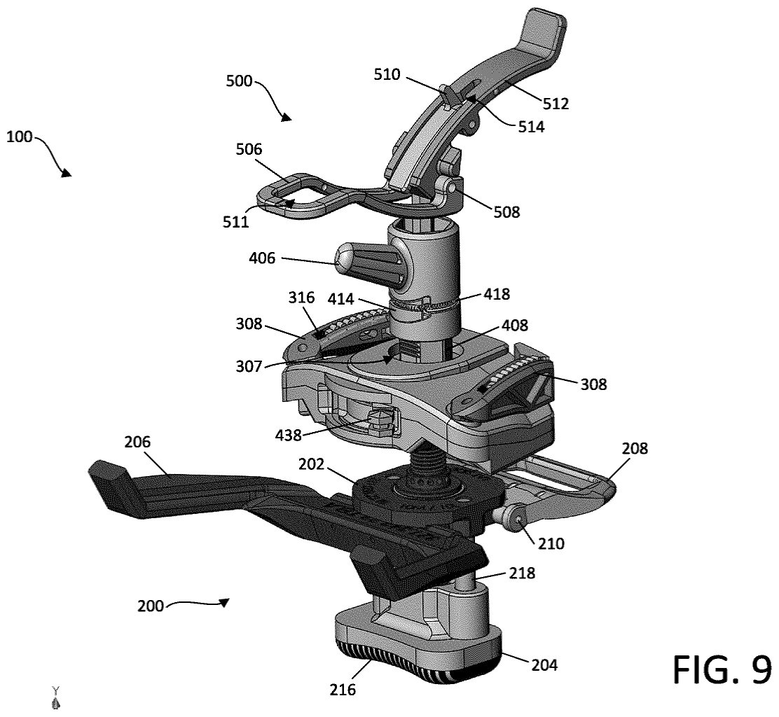

As shown in FIGS. 9-14, among others, once the puck 100 is positioned at the desired location on the subject, the puck 100 may transition from the orientation position to the extended position. In some embodiments, the handle 500 may receive a strap. For example, a strap maybe disposed on the outer handle 506 and the catch 510 of the handle 500. To position the strap properly for tensioning, the outer handle 506 may move from the receiving position to the first rotation position. With the outer handle 506 in the rotation position, the strap may move from the handle 500 to the fin 406. With the strap on the fin 406, the strap may be extending from the subject on a first side of the puck 100, over a first edge the chassis 300, on top of the fin 406, over a second edge of the chassis 300, and back to the subject on a second side of the puck 100. The puck 100 may have two strap retention mechanisms 308 configured to retain the position of the strap relative to the chassis 300. For example, a first strap retention mechanism 308 on a first side of the chassis 300 may engage a first portion of the strap and lock into the latched position via a first keeper 312. A second strap retention mechanism 308 on a second side of the chassis 300 may engage a second portion of the strap and lock into the latched position via a second keeper 312. The strap may be held stable (as shown in FIG. 9) by a combination of strap retention mechanisms 308 and wings 206, 208. The latches 314 may slide within the strap retention mechanisms 308 and may be forced by a latch spring 316 to lock the strap retention mechanisms 308 with the keepers 312.

Referring to FIGS. 8, 12, and 13, among others, the tensioning and extension means of the puck 100 are shown, according to an example embodiment. The ratchet 400 may be configured to facilitate the tensioning and extending of the puck 100. FIG. 8 shows the ratchet 400 in a retracted position. FIGS. 9, 11, 12, and 13, among others, show the ratchet 400 in an extended position.

The body 402 may be slidably coupled with the spool 408. The body 402 may be manually moved from a lower position (as shown in FIG. 8) to an upper position (as shown in FIG. 12). The body 402 may be moved to the upper position before the strap is installed. The spool groove 410 may be configured to prevent downward motion of the fin 406 and the body 402 when the strap provides an axial load on the puck 100. For example, the lock 414 may be configured to engage with at least one spool groove 410 to prevent the downward motion of the body 402 and the fin 406. The garter spring 418 can be disposed in the spring groove 416. The garter spring 418 can keep the lock 414 in contact with the spool groove 410 to maintain the axial locking under the load from the strap. The garter spring 418 can prevent the downward motion of the body 402 relative to the spool 408, but may also allow upward motion of the body 402 relative to the spool 408, such that the spool 408 can slide out of the body 402.

With the strap locked in position via the strap retention mechanisms 308, the handle 500 may rotate to initiate the tensioning of the strap. The drive post 504 may be slidably coupled with the primary screw 440. Torque may be transferred from the handle 500 to the primary screw 440 and to the shear pins 448. With the torque below a torque threshold, the torque may be transferred from the primary screw 440 to the spool 408, to the body 402, and ultimately to the fin 406. The torque may cause the fin 406 to rotate around an axial axis 600 of the puck 100. Rotation of the fin 406 may cause the strap to wind around the body 402 to tension the strap (e.g., pre-tension the strap, remove slack from the strap). When pressure is released from the handle, the spool 408 has radial ratchet teeth 428 that can interact with a pawl 430 to prevent anti-rotation of the spool 408 and fin 406.

When the strap is sufficiently tightened beyond the torque threshold of the shear pins 448, the shear pins 448 may break or fail, which may facilitate rotation of the primary screw 440 in relation to the spool 408 to cause extension of the ratchet 400 and the overall puck 100. For example, the primary screw 440 may be threadedly coupled with the spool 408 such that rotation of the primary screw 440 may cause the primary screw 440 to translate in an axial direction relative to the spool 408 such that at least part of the first portion 444 of the primary screw 440 is disposed outside of (e.g., below) the spool 408 and the chassis 300.

The secondary screw 452 may be rigidly coupled to the primary screw 440. The primary flange 442 and the secondary flange 460 may encapsulate the bearing 214 of the upper profile 202. As such, the extension of the primary screw 440 may also rotate the secondary screw 452 and extend the upper profile 202 and the wings 206, 208 downward into the injured subject. The rotation of the secondary screw 452, which may have a left hand thread, may advance a nut 456. The nut 456 may be rigidly coupled to the lower profile 204 such that the nut 456 does not rotate relative to the lower profile 204. The shoulder screw 218 of the base assembly 200 may prevent the lower profile 204 from rotating with the secondary screw 452, but also allow the lower profile 204 to slide relative to the upper profile 202. Therefore, the lower profile 204 and the pressure surface 216 may extend away from the upper profile 202 and toward the subject responsive to the rotation of the secondary screw 452. The secondary screw 452 may be configured to unscrew from the nut 456. For example, at a predetermined distance (e.g., approximately ¾ inch of extension of the lower profile 204 in relation to the upper profile 202), the secondary screw 452 may unscrew from the nut 456.

Primary screw 440 may continue to move and cause the upper profile 202, wings 206, 208, and lower profile 204 to continue extending toward the body from the fixed strap until the an artery is fully occluded and bleeding stops. Since the lower profile 204, upper profile 202, and wings 206, 208 can advance together, stability may be maintained. For example, if the lower profile 204 advanced alone, the wings 206, 208 may loose contact with the subject an reduce stability. This two-screw mechanism (e.g., the primary screw 440 and the secondary screw 452) may provide a more compact and more stable series of events in that initial strap tension occurs with the lower profile 204 closest to the fin 406 and allows the wings 206, 208 to firmly seat themselves before the lower profile 204 is extended towards the body, thus eliminating an instability event.

In some embodiments, the puck 100 can be removed or repositioned by loosening the handle 500 and flipping the lever 438. Upon unscrewing motion or loosening the handle 500, the nut 456 and lower profile 204 may retract in relation to the upper profile 202 until the secondary screw 452 screws into the nut 456 and compresses the wave spring 458. The nut 456 may unscrew onto the shaft of the secondary screw 452. In this way, wave spring 458 facilitates reengagement of the nut 456 with the threaded portion 462 of the secondary screw 452 and facilitates further unscrewing of the primary screw 440. When the primary screw 440 is unscrewed all the way, the lever 438 can release the body 402 and the fin 406 such that the strap can become loose.

### Assembly of the Puck 100

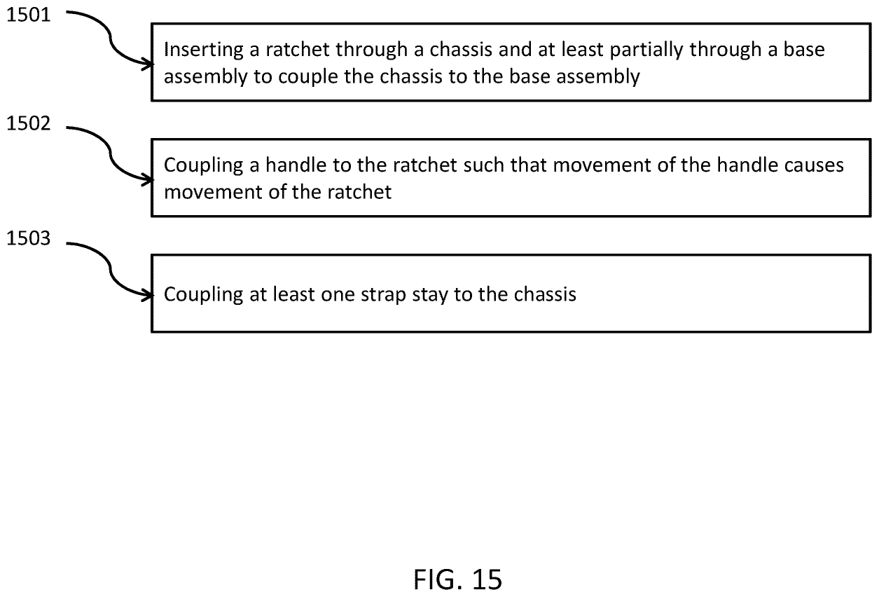

A method of assembling a pressure application system is provided. FIG. 15 is a flowchart depicting a method for assembling a pressure application system. At step 1501, the chassis is coupled tot the base assembly such that a ratchet extends through the chassis and at least partially in the base assembly. The base assembly can include an upper profile and a lower profile. The chassis can be movably coupled with the base assembly to allow movement of the base assembly relative to the chassis. The ratchet can be movably coupled with the chassis to allow movement of the chassis relative to the ratchet. The step 1501 can further comprise inserting the ratchet through a chassis aperture of the chassis. The step 1501 can further comprise coupling the lower profile with the upper profile of the base assembly. The step of coupling the lower profile with the upper profile can comprise inserting the ratchet at least partially through a base aperture extending through the upper profile of the base assembly. The ratchet can be inserted at least partially through a profile pocket defined by the lower profile of the base assembly. Prior to the step 1501, an upper chassis and a lower chassis can be coupled to each other to a form the chassis.

The ratchet can comprise a body defining a body aperture extending axially through at least a portion of the body. The method of assembling the pressure application system can further comprise coupling a spool with the body of the ratchet. The step of coupling the spool with the body of the ratchet can further comprise inserting the spool at least partially through the body aperture. The ratchet can include at least one primary screw. A portion of the primary screw of the ratchet can be coupled with the spool. The step of coupling a portion of the primary screw of the ratchet with the spool can further comprise threading the portion of the primary screw into a spool aperture of the spool.

At step 1502, a handle can be coupled to the ratchet such that movement of the handle causes movement of the ratchet. The handle can include a drive post and a handle portion coupled with the drive post. The step 1502 can further comprise coupling the handle portion with the drive post. The step 1502 can further comprise coupling the drive post with the primary screw. The drive post can be slidably coupled with the primary screw. Additionally or alternatively, the ratchet can include at least one secondary screw that can be fixedly coupled to the primary screw. The primary screw can further comprise a screw aperture extending axially through the primary screw. The secondary screw can be disposed in the screw aperture, such that a rotation of the drive post translates the secondary screw.

At step 1503, at least one strap retention mechanism can be coupled to the chassis. The method of assembling the pressure application system can further comprise applying a shear pin. The shear pin can extend between a portion of the ratchet and a component of the pressure application system. The method of assembling the pressure application system can further comprise coupling at least one stabilizing component (e.g., a medial wing, lateral wing) to the upper profile of the base assembly. The method of assembling the pressure application system can further comprise coupling a latch to each strap retention mechanism. The method of assembling the pressure application system can further comprise coupling at least one engagement feature to the chassis.

### Implementation of the Puck 100

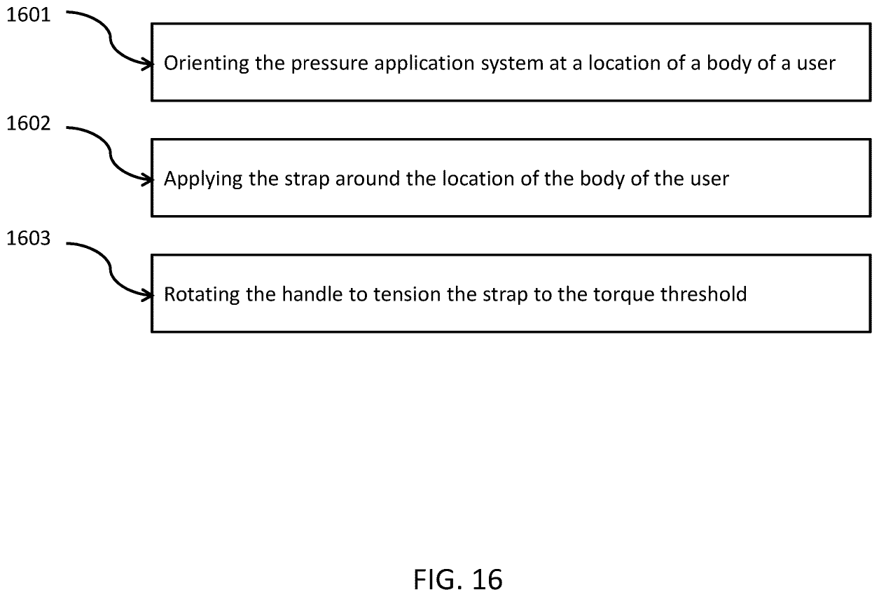

A method of using a pressure application system is provided. FIG. 16 is a flowchart depicting a method for using a pressure application system. At step 1601, a pressure application system can be oriented at a location of a body of a user. The pressure application system can comprise a base assembly, a chassis coupled to the base assembly, a ratchet, a handle coupled with the ratchet, and at least on strap retention mechanism coupled to the chassis. The base assembly can include an upper profile and a lower profile. The ratchet can extend through the chassis and at least partially in the base assembly. A movement of the handle can move the ratchet. The at least one strap retention mechanism can be configured to receive a strap. The pressure application system can further comprise at least one stabilizing component (e.g., a medial wing 206, lateral wing 208) coupled to the upper profile of the base assembly. The method of using the pressure application system can further comprise moving the at least one stabilizing components from a stowed position to a deployed position.

At step 1602, a strap can be applied around the location of the body of the user. The step 1602 can further comprise extending the strap over a fin extending from a surface of the pressure application system. A strap can be extended from a first side of the pressure application system, over the fin, to a second side of the pressure application system. The first side of the pressure application system can be a first side of the chassis. A first portion of the strap can be engaged with a first strap retention mechanism disposed on the first side of the chassis. The second side of the pressure application system can be a second side of the chassis. A second portion of the strap can be engaged with a second strap retention mechanism disposed on the second side of the chassis. The first strap retention mechanism can be engaged with a first engagement feature to retain the first strap retention mechanism in a latched position to maintain the position of the strap. The second strap retention mechanism can be engaged with a second engagement feature to retain the second strap retention mechanism in a latched position to maintain the position of the strap. A latch can be coupled with each of the strap retention mechanisms. The latch of each strap retention mechanism can be engaged with its respective engagement feature.

At step 1603, the handle can be rotated to tension the strap. The step 1603 can further comprise rotating the handle up to a torque threshold. The torque threshold can be determined by physical properties of a shear pin disposed within the pressure application system. Rotating the handle below the torque threshold, can rotate a fin extending from a surface of the ratchet such that the strap winds around the body of the ratchet and tensions the strap. The step 1603 can further comprise rotating the handle beyond the torque threshold causing the shear pin to fail or break causing the ratchet to extend. The method of using the pressure application system can further comprise reversibly rotating the handle and flipping a lever to release the tensioning of the strap.

It should be noted that there may be many other embodiments that would provide a tensioning and extension means with a stability means. For example, wings 206, 208 may be coupled directly to the chassis 300 instead of an intermediate upper profile 202, or a gas spring extension means could replace the screw extension means of the ratchet 400. Similarly, a pressure bladder with wings could have a tensioning means to realize a configuration where improvements to the puck 100 allow pre tensioning of a strap, extending the pressure surface into the junctional area, and stabilizing the orientation of the pressure surface to the body and the strap.

What is claimed is:

1. A pressure application system, comprising:
a base assembly including an upper profile and a lower profile;
a chassis coupled to the base assembly;
a ratchet extending through the chassis and at least partially into the base assembly;
a fin extending from a surface of the ratchet;
a handle coupled with the ratchet, the handle engaging a primary screw of the ratchet; and
at least one strap retention mechanism coupled to the chassis, the at least one strap retention mechanism configured to receive and retain a strap;
wherein rotation of the handle below a torque threshold causes the fin to rotate and wind the strap around the ratchet to tension the strap, and rotation of the handle above the torque threshold translates the ratchet axially to extend the lower profile away from the upper profile.

2. The pressure application system of claim 1, the chassis movably coupled with the base assembly to allow movement of the base assembly relative to the chassis.

3. The pressure application system of claim 1, the ratchet movably coupled with the chassis to allow movement of the chassis relative to the ratchet.

4. The pressure application system of claim 1, the chassis further comprising a chassis aperture configured to receive at least a portion of the ratchet.

5. The pressure application system of claim 1, the chassis further comprising at least one engagement feature configured to engage the at least one strap retention mechanism to retain the strap retention mechanism in a latched position.

6. The pressure application system of claim 5, the at least one strap retention mechanism further comprising a latch, the latch configured to be received by the at least one engagement feature.

7. The pressure application system of claim 1, the ratchet comprising a body defining a body aperture extending axially through at least a portion of the body.

8. The pressure application system of claim 7, further comprising a spool disposed at least partially in the body aperture.

9. The pressure application system of claim 8, the spool further comprising a spool aperture extending axially therethrough, wherein the primary screw is partially disposed in the spool aperture.

10. The pressure application system of claim 9, the handle further comprising a handle portion coupled to a drive post, the drive post extending through the body aperture to engage the primary screw, wherein a rotation of the handle above the torque threshold rotates the drive post and translates the primary screw in an axial direction.

11. The pressure application system of claim 10, the primary screw further comprising a screw aperture extending axially therethrough, wherein a secondary screw of the ratchet is disposed in the screw aperture, and wherein a rotation of the handle above the torque threshold rotates the drive post and translates the secondary screw in an axial direction.

12. The pressure application system of claim 9, the primary screw further comprising a shear pin configured to prevent rotation of the primary screw relative to the spool until a force applied to the shear pin reaches the torque threshold.

13. The pressure application system of claim 1, further comprising a shear pin disposed at least partially within the ratchet, wherein the torque threshold is determined by the shear pin.

14. The pressure application system of claim 1, the base assembly further comprising a base aperture configured to receive at least a portion of the ratchet.

15. A method of using a pressure application system, comprising:
orienting the pressure application system at a location of a body of a user, the pressure application system, comprising:
a base assembly including an upper profile and a lower profile;
a chassis coupled to the base assembly;
a ratchet extending through the chassis and at least partially into the base assembly;
a fin extending from a surface of the ratchet;
a handle coupled with the ratchet, the handle engaging a portion of the ratchet such that rotation of the drive post translates the portion of the ratchet in an axial direction; and
at least one strap retention mechanism coupled to the chassis, the at least one strap retention mechanism configured to receive and retain a strap;
wherein rotation of the handle below a torque threshold causes the fin to rotate and wind the strap around the ratchet to tension the strap, and rotation of the handle above the torque threshold causes the ratchet to translate axially to extend the lower profile away from the upper profile;
applying the strap around the location of the body of the user; and
rotating the handle to tension the strap to the torque threshold.

16. The method of claim 15, further comprising, rotating the handle beyond the torque threshold to extend the ratchet.

17. The method of claim 15, further comprising reversibly rotating the handle and flipping a lever to release the tensioning of the strap.

18. The method of claim 15, wherein applying the strap around the location of the body of the user further comprises:
extending a strap from a first side of the chassis, over the fin, to a second side of the chassis.

19. The method of claim 18, wherein applying the strap around the location of the body of the user further comprises:
engaging a first portion of the strap with a first strap retention mechanism disposed on the first side of the chassis; and
engaging a second portion of the strap with a second strap retention mechanism disposed on the second side of the chassis.

20. The method of claim 19, further comprising:
engaging the first strap retention mechanism with a first engagement feature to retain the first strap retention mechanism in a latched position; and
engaging the second strap retention mechanism with a second engagement feature to retain the second strap retention mechanism in a latched position.
